(19) United States
(12) Patent Application Publication
(10) Pub. No.: US 2020/0407711 A1
(43) Pub. Date: Dec. 31, 2020

(54) SYSTEMS AND METHODS FOR SCORING RESULTS OF IDENTIFICATION PROCESSES USED TO IDENTIFY A BIOLOGICAL SEQUENCE

(71) Applicant: Advanced Molecular Diagnostics, LLC, Montvale, NJ (US)

(72) Inventors: Daniel R. COHEN, Monsey, NY (US); Pablo J. UMANSKY, Spring Valley, NY (US)

(21) Appl. No.: 16/905,282

(22) Filed: Jun. 18, 2020

Related U.S. Application Data
(60) Provisional application No. 62/868,563, filed on Jun. 28, 2019.

(51) Int. Cl. C12N 15/10; C40B 50/06; C12Q 1/6806; C12Q 1/6869

(52) U.S. Cl. CPC C12N 15/1068; C40B 50/06; C12Q 2535/101; C12Q 1/6869; C12Q 2535/122; C12Q 1/6806

(57) ABSTRACT

Systems, methods, and machine-readable instructions stored on machine-readable media are disclosed for receiving a first DNA sequencing result and a second DNA sequencing result, wherein the first DNA sequencing result is a result of a first DNA sequencing technique and the second DNA sequencing result is a result of a second DNA sequencing technique. A difference is determined between the first DNA sequencing result and the second DNA sequencing result. Parameters are scored corresponding to the first DNA sequencing result and to the second DNA sequencing result, wherein the scoring includes: determining a value range of a parameter in a set of reference parameters corresponding to a value of a corresponding parameter of the parameters; and assigning a score associated with the value range of the parameter in the set of reference parameters as a score of the corresponding parameter. A determination is made that the first DNA sequencing result or the second DNA sequencing result is conclusive or inconclusive based on respective scores of the parameters of the first DNA sequencing result and the second DNA sequencing result. An indication is made to perform a third DNA sequencing using a third DNA sequencing technique when the first DNA sequencing result or the second DNA sequencing result is inconclusive.

### CROSS-REFERENCE TO RELATED APPLICATIONS

This application claims the benefit of the filing date of, and priority to, U.S. Provisional Application No. 62/868,563, filed Jun. 28, 2019, the entire contents of which is hereby incorporated herein by reference.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### Field of Disclosure

The present disclosure generally relates to systems and methods of scoring results of different identification processes, more particularly to increasing the efficiency of computer processing of DNA sequence data identification.

### BACKGROUND

### Description of the Related Art

Cancer

Cancer is a disease in which unhealthy cells divide abnormally and invade healthy cells. To detect the presence of cancer, DNA sequencing techniques may be used to identify genetic variants or mutations in a patient's DNA or to spot for other oncologic precursors. Mutations may be pathogenic (harmful) or benign (non-harmful). Thus, it is crucial for DNA sequencing results to be accurate if a physician is to form the correct diagnosis.

### SUMMARY

A system of one or more computers can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system including: a non-transitory memory, and one or more hardware processors coupled to the non-transitory memory to execute instructions from the non-transitory memory to perform operations including: receiving a first DNA sequencing result and a second DNA sequencing result, wherein the first DNA sequencing result is a result of a first DNA sequencing technique and the second DNA sequencing result is a result of a second DNA sequencing technique; determining a difference between the first DNA sequencing result and the second DNA sequencing result; scoring parameters corresponding to the first DNA sequencing result and to the second DNA sequencing result, wherein the scoring includes: determining a value range of a parameter in a set of reference parameters corresponding to a value of a corresponding parameter of the parameters; and assigning a score associated with the value range of the parameter in the set of reference parameters as a score of the corresponding parameter; determining that the first DNA sequencing result or the second DNA sequencing result is conclusive or inconclusive based on respective scores of the parameters of the first DNA sequencing result and the second DNA sequencing result; and indicating to perform a third DNA sequencing using a third DNA sequencing technique when the first DNA sequencing result or the second DNA sequencing result is inconclusive. Other examples of this aspect include corresponding computer systems, apparatus, and computer programs recorded on one or more computer storage devices, each configured to perform the actions of the methods.

One general aspect includes a method including: receiving a first DNA sequencing result and a second DNA sequencing result, wherein the first DNA sequencing result is a result of a first DNA sequencing technique and the second DNA sequencing result is a result of a second DNA sequencing technique; determining a difference between the first DNA sequencing result and the second DNA sequencing result; scoring parameters corresponding to the first DNA sequencing result and to the second DNA sequencing result, wherein the scoring includes: determining a value range of a parameter in a set of reference parameters corresponding to a value of a corresponding parameter of the parameters; and assigning a score associated with the value range of the parameter in the set of reference parameters as a score of the corresponding parameter; determining that the first DNA sequencing result or the second DNA sequencing result is conclusive or inconclusive based on respective scores of the parameters of the first DNA sequencing result and the second DNA sequencing result; and indicating to perform a third DNA sequencing using a third DNA sequencing technique when the first DNA sequencing result or the second DNA sequencing result is inconclusive. Other examples of this aspect include corresponding computer systems, apparatus, and computer programs recorded on one or more computer storage devices, each configured to perform the actions of the methods.

One general aspect includes a non-transitory machine-readable medium having stored thereon machine-readable instructions executable to cause at least one machine to perform operations including: receiving a first DNA sequencing result and a second DNA sequencing result, wherein the first DNA sequencing result is a result of a first DNA sequencing technique and the second DNA sequencing result is a result of a second DNA sequencing technique; determining a difference between the first DNA sequencing result and the second DNA sequencing result; scoring parameters corresponding to the first DNA sequencing result and to the second DNA sequencing result, wherein the scoring includes: determining a value range of a parameter in a set of reference parameters corresponding to a value of a corresponding parameter of the parameters; and assigning a score associated with the value range of the parameter in the set of reference parameters as a score of the corresponding parameter; determining that the first DNA sequencing result or the second DNA sequencing result is conclusive or inconclusive based on respective scores of the parameters of the first DNA sequencing result and the second DNA sequencing result; and indicating to perform a third DNA sequencing using a third DNA sequencing technique when the first DNA sequencing result or the second DNA sequencing result is inconclusive. Other examples of this aspect include corresponding computer systems, apparatus, and computer programs recorded on one or more computer storage devices, each configured to perform the actions of the methods.

### BRIEF DESCRIPTION OF THE DRAWINGS

The following drawings form part of the present specification and are included to demonstrate certain aspects of the present invention. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1 is an organizational diagram illustrating a system for analyzing and scoring results from a plurality of DNA sequencing techniques and determining whether a sequencing result is true; and

FIG. 2 is a flow diagram illustrating a method for analyzing and scoring results from a plurality of DNA sequencing techniques and determining whether a sequencing result is true.

### DETAILED DESCRIPTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

In the following description, specific details are set forth describing some examples consistent with the present disclosure. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional.

DNA is made up of a long string of nucleotides of DNA. In humans, DNA is divided into 24 linear molecules or genes. The longest human gene comprises 260 million nucleotides, and the shortest 50 million nucleotides. Altogether, the human genome is made up of over 3 billion nucleotides. Each nucleotide includes a nucleoside and at least one phosphate group. A nucleoside is comprised of a nitrogenous base, such as adenine (A), cytosine (C), guanine (G), and thymine (T) in DNA or and Uracil (U) in RNA and a five-carbon sugar (ribose in RNA or deoxyribose in DNA).

In healthy DNA, the nucleobases may be arranged in a particular sequence at a particular location in the DNA, e.g., ATTGTT at base pairs 43676342 to 43676347. In some examples, the location may be more generally expressed using a cytogenic location, such as 17q21.31. The band 17q21.31 contains approximately 4 million base pairs, spanning from base pairs 43044295 to 43125483. The first number, here 17, refers to the chromosome number on which the gene can be found. The letter p or q refers to the short or long arm, respectively, of the chromosome. The last number refers to the position of the gene on the chromosome. The position of a gene is based on a distinctive pattern of light and dark bands that appear when the chromosome is stained in a certain way. The position is usually designated by two digits (representing a region and a band), which are sometimes followed by a decimal point and one or more additional digits (representing sub-bands within a light or dark area).

For any number of reasons, DNA may undergo a mutation. For example, the base pairs in the example above at 43676342 to 43676347 may change from ATTGTT to ATCGTT. Such a mutation may be benign resulting in no harm to a patient. Other mutations may be pathogenic and cause important functions to be compromised. For example, certain mutations in a BRCA1 gene may cause important tumor-suppressing functions to be lost and lead to the formation of breast cancer.

DNA sequencing techniques include any technique or method to determine the identity and sequence of a nucleobase at any position in the DNA. Thus, DNA sequencing techniques may be used to determine, for example, the nucleobase sequence at base pairs 43676342 to 43676347. DNA sequencing techniques may also be used, for example, to determine some or all of the nucleobase sequence of all 4 million base pairs at 17q21.31, or at any other location in any genome. Examples of “first generation” DNA sequencing technique include techniques based on selective incorporation of chain-terminating dideoxynucleotides, such as in Sanger sequencing; techniques based on partial chemical modification of nucleobases and subsequent cleavage of a DNA strand at bases next to the modified nucleobases, such as in Maxam-Gilbert sequencing. Examples of “second generation” or “next-generation” DNA sequencing (NGS) include techniques based on detection of ions released during polymerization of DNA, such as in ion semiconductor sequencing (ION TORRENT™ ThermoFisher Scientific) and techniques based on reversible termination sequencing, such as in SOLEXA® or ILLUMINA® sequencing products from ILLUMINA, INC. (San Diego, Calif., USA).

Briefly, Ion Torrent™, is a next-generation sequencing methodology that exploits the fact that addition of a dNTP to a DNA polymer releases a hydrogen ion. By measuring the pH change resulting from release of those hydrogen ions, the sequence of a DNA fragment can be determined. Performing the process using semiconductors capable of simultaneously measuring millions of such changes, the determination of the sequence of multiple fragments can be obtained at once in a massively parallel fashion (see e.g., PCT Intl. Pat. Appl. Publ. No. WO2006/110855; U.S. Pat. Appl. Publ. Nos. 2010/0304982, 2010/0300559, 2010/0300895, 2010/0137143, and 2010/0301398, U.S. Pat. Nos. 9,365,904 and 9,598,737, each of which is specifically incorporated herein in entirety by express reference thereto).

Of course, many more sequencing techniques exist, any of which may be used in conjunction with the techniques disclosed herein; such techniques include, but are not limited to, ligation sequencing (SOLiD), colony sequencing, pyrosequencing, combinatorial probe/anchor synthesis (cPAS), DNA nanoball sequencing, heliscope single molecule sequencing, single molecule real time (SMRT) sequencing, nanopore DNA sequencing, and various microfluidics-based systems.

The techniques take advantage of the fact that each sequencing technique introduces peculiarities and artifacts owing to the fundamental chemistry, physics, biology or other science or art upon which the different sequencing techniques were developed. Because of these differences, the sequencing results produced by the various techniques may not always agree, even if the DNA being sequenced using one technique is an identical clone of the DNA being sequenced using another. As disclosed herein, such differences may be used to improve the accuracy, precision, confidence, reliability, or any other desirable trait of a DNA sequencing result. In some examples, such differences are used to identify false positive or false negatives. In some examples, such differences are used to indicate when different sequencing techniques should be used that are more likely to yield a true result. In some examples, such differences are used to determine, which, if any, among the results produced is an accurate one. For example, results produced by techniques based on ion semiconductor sequencing may differ from results produced by techniques based on reversible termination sequencing.

In some examples, a computer program is used to analyze the results produced by each of the different sequencing techniques. For example, parameters such as allele frequency, read quality index, mean coverage, and uniformity of coverage may be analyzed. These parameters are exemplary only, and any other parameters associated with the sequencing process or the sequencing result may be analyzed. In some examples, the DNA sequencing result itself (which may also be considered a parameter) may be analyzed. In some examples, such analysis may be based on whether the sequence in the result is a known mutation. To determine whether a mutation is known and the extent to which it is known, reputable knowledge sources may be consulted, such as medical or genetics textbooks, journals, publications, and databases such as the CLINVAR database maintained by the National Center of Biotechnology Information (NCBI) (Bethesda, Md., USA). Any other public or private source of knowledge may be consulted.

The parameters may be scored based on the analysis. In some examples, scoring proceeds according to a reference scorecard. For example, reference values for parameter X may be divided into several categories. Category A includes values 0 to 10 inclusive, Category B includes values 10 to 20 inclusive, and Category C includes values 20 to 30 inclusive. Each category may be associated with a score. For example, Category A may be associated with a score of 0.7; Category B may be associated with a score of 1.0; and Category C may be associated with a score of 1.3. If the value of parameter X obtained from the sequencing result is 8, the value would fall under Category A. Thus, parameter X's score would be 0.7. In some examples, a score below 1.0 indicates that the result produced by a particular sequencing technique is untrue, a score of 1.0 indicates that the result is inconclusive, and a score above 1.0 indicates that the result is true. In some examples, multiple reference values may be used to indicate degrees of truth, inconclusiveness, and untruth. For example, scores between 0.7 to 0.9 may indicate untruth, 0.9 to 1.1 may indicate inconclusiveness, while 1.1 to 1.4 may indicate truth. The values used here are merely exemplary, and any other values may be used without departing from the spirit of the invention.

Each parameter may be scored, thus the scoring of a plurality of parameters results in a battery of scores. Based on the scores, a decision to perform a third sequencing method may be made. For example, if the scores from a first sequencing technique indicate that the result is untrue, but the scores from a second sequencing technique indicate that the result is true, then the result is inconclusive and a third sequencing technique (e.g., one that is more accurate or less likely to produce artifacts over the same sequence as compared to the first and second sequencing techniques) may be recommended or indicated to determine what the true result is. In some examples, the individual parameters are assigned different weights, such that some heavier-weighted parameters may contribute more to a result's score, and therefore have a greater impact as to the decision to perform the third sequencing method, than other less heavily-weighted parameters.

In some examples, certain individual parameters may function as a counterbalance to the other parameters. For example, metrics such as allele frequency, read quality index, mean coverage, and uniformity of coverage may all indicate that a result is untrue, but if the analysis of knowledge sources indicates that the result is not known to occur, such analysis may result in scores being generated to counterbalance the other parameters' scores and cause the final determination to be that the sequencing result is inconclusive. This may be understood as the combination of the metrics and the analysis of the knowledge source indicating that it is “not known” whether the result is “untrue.” The counterbalancing also works in the opposite direction, i.e., if metrics such as allele frequency, read quality index, mean coverage, and uniformity of coverage all indicate that a result is true, but an analysis of the knowledge sources indicates that the result is not known, then the determination may be that the result is inconclusive, therefore a third sequencing method should be used. Nevertheless, in some examples, the scores of the other parameters may be so indicative of truth or untruth that the weight of the counterbalancing parameters do not result in a determination of uncertainty.

Thus, by analyzing the sequencing results produced by different sequencing techniques and determining if the results are conclusive or not, false positives or false negatives may be avoided. Additionally, determining the conclusiveness of the results in silico means that resequencing may be minimized, thus saving time, labor, and resources.

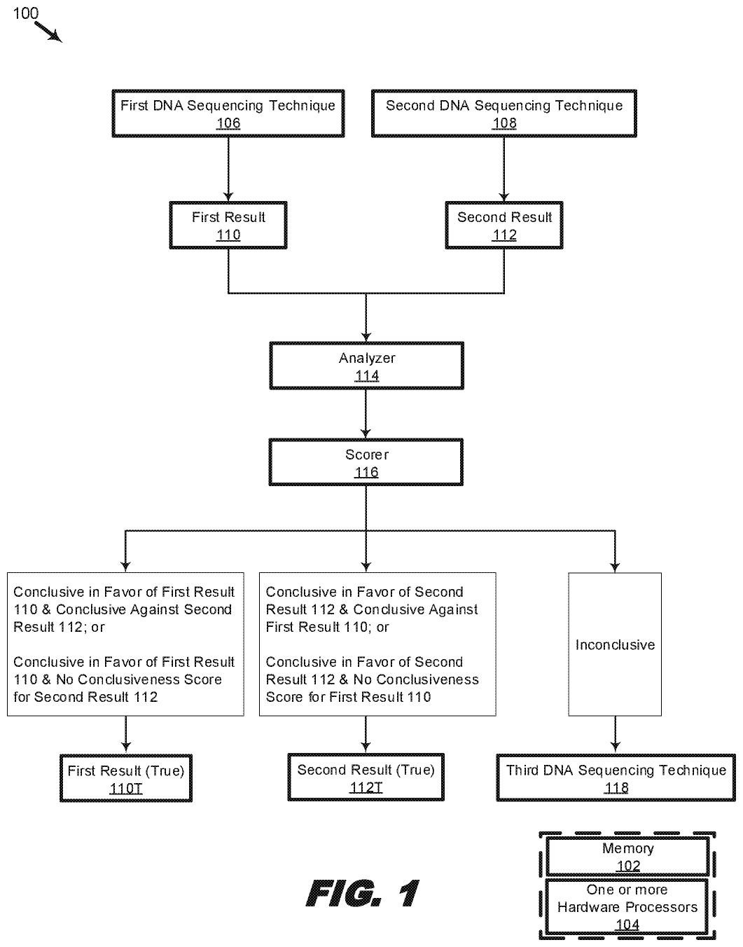

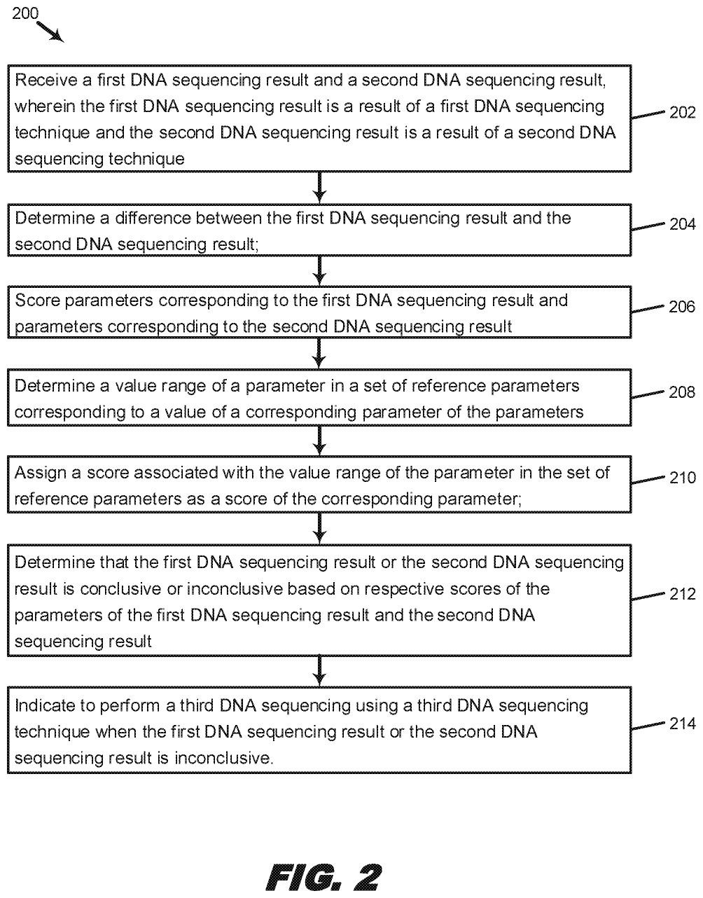

FIG. 1 is an organizational diagram illustrating a system for analyzing and scoring results from a plurality of DNA sequencing techniques, and determining whether a sequencing result is true, in accordance with various examples of the present disclosure. The system 100 includes a non-transitory memory 102 and one or more hardware processors 104 coupled to the non-transitory memory 102. In the present example, the one or more hardware processors 104 executes instructions from the non-transitory memory 102 to perform operations for: receiving a first DNA sequencing result 110 and a second DNA sequencing result 112, wherein the first DNA sequencing result 110 is a result of a first DNA sequencing technique 106 and the second DNA sequencing result 112 is a result of a second DNA sequencing technique 108; determining a difference between the first DNA sequencing result and the second DNA sequencing result; scoring parameters corresponding to the first DNA sequencing result and to the second DNA sequencing result, wherein the scoring includes: determining a value range of a parameter in a set of reference parameters corresponding to a value of a corresponding parameter of the parameters; and assigning a score associated with the value range of the parameter in the set of reference parameters as a score of the corresponding parameter; determining that the first DNA sequencing result or the second DNA sequencing result is conclusive or inconclusive based on respective scores of the parameters of the first DNA sequencing result and the second DNA sequencing result; and indicating to perform a third DNA sequencing using a third DNA sequencing technique 118 when the first DNA sequencing result or the second DNA sequencing result is inconclusive. System 100 may also include additional elements, such as those described herein, and may be used in conjunction with method 200 described with respect to FIG. 2.

Each of the one or more hardware processors 104 is structured to include one or more general-purpose processing devices such as a microprocessor, central processing unit (CPU), and the like. More particularly, a processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. In some examples, each processor is structured to include one or more special-purpose processing devices such as an application specific integrated circuit (ASIC), a field programmable gate array (FPGA), a digital signal processor (DSP), network processor, and so forth. The one or more processors execute instructions for performing the operations, steps, and actions discussed herein.

A non-transitory memory 102 is structured to include at least one non-transitory machine-readable medium on which is stored one or more sets of instructions (e.g., software) including any one or more of the methodologies or functions described herein. The non-transitory memory may be structured to include one or more of a read-only memory (ROM), flash memory, dynamic random access memory (DRAM) such as synchronous DRAM (SDRAM), double data rate (DDR SDRAM), or DRAM (RDRAM), and so forth), static memory (e.g., flash memory, static random access memory (SRAM), and so forth), and a data storage device (e.g., hard disk, magnetic tape, any other magnetic medium, CD-ROM, any other optical medium, RAM, PROM, EPROM, FLASH-EPROM, any other memory chip or cartridge, and/or any other medium from which a processor or computer is adapted to read). Accordingly, any of the operations, steps, and actions of the methods described herein may be implemented using corresponding machine-readable instructions stored on or in a memory that are executable by a processor.

The system 100 includes a bus or other communication mechanism for communicating information data, signals, and information between the non-transitory memory 102, the one or more hardware processors 104, and the various components of system 100. For example, the various components may include a data storage device, which may be local to the system 100 or communicatively coupled to the system 100 via a network interface. Components may further include input/output (I/O) components such as a keyboard, mouse, touch interface, and/or cameras that process user actions such as key presses, clicks, taps, and/or gestures and sends a corresponding signal or instruction to the non-transitory memory 102 for processing by the one or more hardware processors 104 via the bus or other communication pathway. The I/O component may also include an output component such as a display.

In some examples, a user may use the I/O component to command the system 100, via a user interface such as a graphical user interface, a command line interface, or any other interface a user may use to communicate with system 100, to directly or indirectly cause first result 110 and second result 112 to be received by the system 100. First result 110 and second result 112 may include DNA sequencing results such as DNA sequences and any data or parameters associated with sequencing DNA. In some examples, first result 110 may be obtained using a first DNA sequencing technique 106, while second result 112 may be obtained using a second DNA sequencing technique 108. First result 110 may be the same as or different from second result 112, and first DNA sequencing technique 106 may be the same as or different from second DNA sequencing technique 108. In some examples, identical DNA (e.g., DNA clones) is provided to first DNA sequencing technique 106 and second DNA sequencing technique 108.

First DNA sequencing technique 106 and second DNA sequencing technique 108 may include any techniques used to determine the identity and/or order of chemical or biological compounds in any structure. Thus, first DNA sequencing technique 106 and second DNA sequencing technique 108 includes techniques to determine the identity and sequence of nucleobases in helical structures such as ribonucleic acid (RNA) and deoxyribonucleic acid (DNA). Examples of DNA sequencing techniques include any techniques based on: Sanger sequencing; single-nucleotide addition (SNA) sequencing; pyrosequencing; 454 sequencing; cyclic array sequencing; cyclic reversible termination (CRT) sequencing; 3′-O-blocked reversible terminating group sequencing; 3′-unblocked reversible terminating group sequencing; sequencing by hybridization; sequencing-by-synthesis; fluorescent in situ sequencing; single-molecule sequencing, high-throughput sequencing (HTS); next-generation sequencing (NGS); nanopore sequencing; ion semiconductor sequencing; or any other sequencing technology or method.

In some examples, first DNA sequencing technique 106 may include ion semiconductor sequencing, while second DNA sequencing technique 108 may include the use of reversibly-terminated Sanger cleavable fluorescent dideoxynucleotides or reversible termination sequencing. In some examples, the first result 110 and second result 112 may include a post-alignment DNA sequence, i.e., a DNA sequence derived from the alignment of some or all sequencing fragments. For whatever reason, first result 110 may differ from second result 112 even though first DNA sequencing technique 106 and second DNA sequencing technique 108 were provided with identical DNA sequences to sequence. To help a physician determine which, if any, of first result 110 or second result 112 contains a true or accurate reading of the DNA sequence, the first result 110 and second result 112 may be downloaded or otherwise retrieved from their respective DNA sequencers or any other source and provided to system 100.

In some examples, the first result 110 and second result 112 may be retrieved from a computer system associated with the respective DNA sequencers, such as an online or cloud storage system. In some examples, the system 100 may be communicatively coupled by a wired or wireless connection to the DNA sequencing systems, which allows the first result 110 and the second result 112 to be sent from the respective DNA sequencing systems to the system 100 using a local area network (LAN), the internet, or any other communications system. In some examples, the first result 110 and the second result 112 is automatically sent to the system 100 after sequencing is complete. In some examples, a user may manually request for the first result 110 and the second result 112 to be retrieved from the sequencing system or from any other source, such as a local or remote database, a memory, a storage device, or any other software or hardware in which the first result 110 and the second result 112 may be stored. In some examples, a user may retrieve the first result 110 and second result 112 into a storage device and upload the first result 110 and second result 112 into system 100.

In some examples, the system 100 stores the first result 110 and the second result 112 in non-transitory memory 102. The first result 110 and the second result 112 may be analyzed by analyzer 114 and scored by scorer 116. Analyzer 114 and scorer 116 may be any software, hardware, or combination of software and hardware capable of storing and executing instructions to perform the techniques described herein. In some examples, the analyzer 114 or scorer 116 may be separate hardware modules including their own hardware processors and memories. In some examples, analyzer 114 and scorer 116 are integrated into the same hardware module and share a common hardware processor and memory. The hardware processor and memory of analyzer 114 or scorer 116 may be similar to the one or more hardware processors 104 and memory 102. The analyzer 114 or scorer 116 may be coupled to a circuit board via a suitable interface, such as a peripheral component interface (PCI), a USB interface, or any other interface. Such circuit board may house the one or more hardware processors 104 and memory 102 and may include interconnects between each of the interfaces and the one or more hardware processors 104 and memory 102. Thus, instructions or other communications may flow between the analyzer 114, the scorer 116, and the one or more hardware processors 104 and memory 102 via a bus, such as a PCI bus or a system bus.

The memory of analyzer 114 or scorer 116, which may be different from memory 102, may store instructions to perform the techniques described herein. The memory of the analyzer 114 or scorer 116 may also store the sequences to be analyzed. In some examples, the instructions may be executed by a hardware processor local to the analyzer 114 or scorer 116, and the results of the analysis or scoring may be communicated to or provided to system 100 over a bus or a communications network. In some examples, the instructions stored in analyzer 114 or scorer 116 may be provided (e.g., over a bus or a communications network) to the one or more hardware processors 104 for execution.

In some examples, analyzer 114 or scorer 116 may be part of an online- or cloud-based analysis or scoring system. Thus, analyzer 114 and scorer 116 may distribute any instructions or processing tasks across a network of computer systems. Further, analyzer 114 or scorer 116 may perform such processing in parallel using virtualized hardware such as virtual machines. In some examples, the physical machines or virtual machines may execute the instructions in isolated software environments, such as container images.

The instructions for the techniques herein may be coded as, part of, or executable by any general-purpose software or specialized analytical software capable of performing the techniques herein. Such software may be stored in memory 102, or in some examples, in the memory of analyzer 114 or scorer 116. Such software may be packaged as an application, an executable file, an image for execution in an isolated operating environment (e.g., a container), or any other format. In some examples, the software may be a programmable general-purpose spreadsheet or database software, such as MICROSOFT EXCEL or MICROSOFT ACCESS available from Microsoft Corp. (Redmond, Wash., USA). In some examples, the software may be any genetic analysis software, for example, those used to perform single nucleotide polymorphism (SNP) analysis, deletion analysis, duplication analysis, insertion and deletion (indel) analysis, genome validation, exome validation, gene mapping, sequencing analysis, fragment analysis, somatic variant detection, gene fusion detection, cfDNA variant detection, or any other kind of genetic analysis.

The analyzer 114 may receive, retrieve, generate or analyze the following parameters of a DNA sequence from first result 110 and second result 112: variant localization parameters; nucleotide change parameters; in silico parameters; clinical parameters; and sequencing metrics parameters.

Variant localization parameters may include, for example: chromosome number; chromosome coordinate; and gene identifier. The chromosome number parameter may include the number of the chromosome corresponding to the sequencing result. For example, in a human, a genetic mutation in chromosome number 17 is known to be responsible for breast cancer. The chromosome coordinate parameter may include the base pair location of the chromosome in the sequencing result. The chromosome coordinate may also be based on any other system for identifying a location on a chromosome, such as cytogenic location, genomic location, Human Genome Variation Society (HGVS) sequence variant nomenclature, etc. In some examples, the chromosome coordinate includes a starting base pair and an ending base pair, such as 43676342 to 43676347. The gene identifier parameter may include the name of a unique sequence of nucleotides, such as the BRCA1 gene.

Thus, in some examples, the analyzer 114 may determine from corresponding data in first result 110 or second result 112 that the sequence in first result 110 and second result 112 is, for example, a sequence of the BRCA1 gene located between base pairs 43676342 and 43676347 on chromosome 17. In some examples, such as where the variant localization parameters are not included in first result 110 or second result 112, analyzer 114 may determine such information by identifying the sequence in first result 110 or second result 112 in medical literature, medical databases, or any other knowledge source, and retrieving the corresponding variant localization parameters from such knowledge sources.

Each variant localization parameter may be stored in a data structure such that the variant localization parameters correspond to the other parameters in the data structure (e.g., nucleotide change parameters, clinical parameters, and/or any other parameters) pertaining to the same sequence or variant. Examples of data structures include: tables, databases, arrays, linked list, record, union, tagged union, object, stacks, queues, hash tables, trees, graphs, etc.

Nucleotide change parameters may include, for example: mutation type; sequence type; genotype; consequence; and homopolymer length. The mutation type parameter may indicate the type of mutation present in a sequence, such as, SNPs, deletions, duplications, indels, or any other change or mutation to a DNA sequence. For example, the analyzer 114 may identify a SNP from A to T at base pair 43676342 in first result 110 but not second result 112.

The sequence type parameter may indicate whether a sequence is a reference type sequence or a variant type sequence. In some examples, the analyzer 114 may determine that part or all of a sequence is of a reference type if a corresponding part or all of the sequence does not include any mutations. In some examples, the analyzer 114 may determine that part or all of a sequence is of a variant type if a corresponding part or all of the sequence includes mutations. To determine whether a mutation exists, the analyzer 114 may compare the sequences in the first result 110 and the second result 112 with normal or healthy sequences in medical literature, public or private medical databases, prior sequences from the same patient, sequences from healthy patients, etc.

The genotype parameter may include the zygosity of the genotype, such as homozygous, heterozygous, hemizygous, nullizygous, etc. In some examples, the analyzer 114 may determine zygosity based on a comparison of the alleles of a chromosome pair at any locus.

The consequence parameter may include the consequence of the mutation. For example, a mutation may cause an intron variant, a synonymous variant, a missense variant, an upstream gene variant, a downstream gene variant, a 5′ untranslated (UTR) variant, an in-frame, a frame shift, a stop gained, etc. The analyzer 114 may determine the consequence of a mutation by analyzing the reading frames of each sequence in the first result 110 and second result 112, determining where the mutation has occurred, and the impact of the mutation of the reading frame at loci of interest.

The homopolymer length parameter may be used to indicate, for example, if a homopolymer length in a variant is below or exceeds a predetermined threshold. For example, if the threshold is set to 5 repeating units, the analyzer 114 may determine whether the homopolymer length in first result 110 or second result 112 is equal to or longer than 5 repeating units, the number of homopolymer lengths in first result 110 or second result 112 equal to or exceeding 5 repeating units, the number of homopolymer lengths in first result 110 or second result 112 less than 5 repeating units, etc. The results of the analyzer's 114 determination may be captured in fields corresponding to the nucleotide change parameters in a data structure such as a table or database. Each nucleotide change parameter may be captured in a data structure such that the nucleotide change parameters correspond to the other parameters in the data structure (e.g., variant localization parameters, clinical parameters, and/or any other parameters) pertaining to the same sequence or variant.

In silico parameters may include, for example, the determination as to whether a particular mutation is pathogenic or benign based on evaluation performed by any software, such as: MUTATIONTASTER, available at www.mutationtaster.org, further described in Schwarz et al., (2014); POLYPHEN, available at http://genetics.bwh.harvard.edu/pph2/index.shtml, and further described in Adzhubei et al., (2010); and SIFT, available at https://sift.bii.a-star.edu.sg/, further described in Vaser et al., (2016) and Sim et al., (2012).

In some examples, the analyzer 114 may upload any detected variants for in silico evaluation performed by software such as the above. The results of the evaluation may be independently captured in fields corresponding to the in silico parameters in a data structure such as a table or database. For example, MUTATIONTASTER may indicate that a part of a sequence in first result 110 is pathogenic, but the corresponding sequence in second result 112 is benign. However, POLYPHEN may indicate the opposite, and SIFT may indicate something different altogether. Each in silico parameter may be captured in a data structure such that the in silico parameters correspond to the other parameters in the data structure (e.g., variant localization parameters, nucleotide change parameters, and/or any other parameters) pertaining to the same sequence or variant.

Clinical parameters may include, for example, reference SNP identification number (rs ID number), annotations, and publication. The rs ID number parameter may include an identification tag assigned by NCBI to a group (or cluster) of SNPs that map to an identical location. In some examples, the analyzer 114 may determine if the SNP or other mutation in the variant detected in the first result 110 or the second result 112 has already been submitted to an NCBI database, and if so, the analyzer 114 may retrieve the rs ID number from such database.

The annotation parameter may include annotations used by public or private databases storing genetic information, such as the CLINVAR database. In some examples, the annotation parameter may include “benign polymorphism,” “uncertain significance,” “pathogenic,” or “not listed.” In some examples, analyzer 114 may access the CLINVAR database to retrieve annotations of variants detected in the first result 110 or the second result 112 from matching variants in the CLINVAR database.

The publication parameter may be used to indicate whether a variant detected in the first result 110 or the second result 112 is known in medical literature or has been reported anywhere in any form by any reputable source. In some examples, analyzer 114 may search medical literature, libraries, or any other information source for the detected variant using any of the variant's parameters, such as the variant's sequence as reported in first result 110 or second result 112, the variant's localization parameters, the variant's nucleotide change parameters, etc. If the analyzer 114 finds information about the variant detected in the first result 110 or the second result 112 in an information source, the analyzer 114 may retrieve relevant information from the information source, such as the name of the publisher, publication date, author, publication title, etc. Each clinical parameter may be captured in a data structure such that the clinical parameters correspond to the other parameters in the data structure (e.g., variant localization parameters, nucleotide change parameters, and/or any other parameters) pertaining to the same sequence or variant.

Sequencing metrics parameters may include, for example: allele frequency; allele coverage; read quality index; total length of amplicon region; total percentage of reads passing filter (% PF reads); total aligned reads; percent aligned reads; total probe bases; total aligned non-probe bases; total percentage of bases passing filter PF (% PF bases); percent Q30 bases; total aligned bases; percent aligned bases; mismatch rate; amplicon mean coverage; and uniformity of coverage. Each sequencing metrics parameter may be captured in a data structure such that the sequencing metrics parameters correspond to the other parameters in the data structure (e.g., variant localization parameters, nucleotide change parameters, and/or any other parameters) pertaining to the same sequence or variant.

The allele frequency parameter may include a frequency with which an allele (e.g., a variant allele) is detected in the first result 110 or the second result 112. In some examples, the allele frequency parameter is included in the first result 110 or second result 112. In some examples, the allele frequency is calculated by analyzer 114.

The allele coverage parameter may include a measurement (e.g., count, percentage, etc.) of sequenced alleles and the directionality of allele coverage (e.g., forwards or reverse) with respect to the sequencing direction. In some examples, the sequencing direction is 5′-to-3′. In some examples, the sequencing direction is 3′-to-5′. In some examples, the allele coverage parameter may be included in first result 110 or second result 112. In some examples, the analyzer 114 measures the sequenced alleles and the directionality of the allele coverage.

The read quality index parameter may include quality scores that measure the probability that a base is called incorrectly. The quality scores may be expressed as Q10, Q20, or Q30, where Q=−10 log₁₀(P), and where P is the estimated probability of the base call being wrong. The probability of an incorrect base call in Q10 is 1 in 10; in Q20, 1 in 100; in Q30, 1 in 1000. The inferred base call accuracy in Q10 is 90%, Q20 99%, and Q30 99.9%. In some examples, quality scores may be calculated using the Phred algorithm, for example, as described in Ewing et al. (1998). In some examples, the quality scoring algorithm analyzes sequencing metrics, such as electropherogram peak heights and shape or other parameters relevant to sequencing chemistry obtained from empirical data sets of known sequence accuracy, against multivariate lookup tables to calculate a quality score. In some examples, the read quality index may be included in the first result 110 or second result 112. In some examples, the read quality index may be calculated by analyzer 114 by processing the first result 110 or second result 112 using the quality scoring algorithm. The output of such processing may be a quality score of, for example Q30. In some examples, the analyzer 114 reports the Q score as the read quality index parameter.

The percent Q30 bases parameter may include a percentage of bases that have a Q score equal to or higher than Q30. In some examples, the Q score of each base is included in the first result 110 or second result 112. In some examples, the analyzer 114 calculates the Q score of each base as described above. In some examples, the analyzer 114 divides the number of bases having a Q score equal to or higher than 30 by the total number of bases, multiplied by 100%, to obtain the percent Q30 bases parameter.

The length of amplicon region parameter may include a number of bases in the amplified region. In some examples, the length of amplicon region parameter may be included in first result 110 or second result 112. In some examples, the length of amplicon region parameter includes the length of one, some, or all amplicon regions in a variant detected by the analyzer 114 in the first result 110 or second result 112. In some examples, the length of an amplicon region may be included in the first result 110 or second result 112. In some examples, the length of an amplicon region may be calculated based on the number of bases between the amplicon start and end markers.

The total percentage of reads passing filter (% PF reads) parameter may include a percentage of clusters that pass a chastity filter. In some examples, the % PF reads parameter may be included in first result 110 or second result 112. % PF reads may indicate the purity of a signal of a one or more clusters of identical DNA sequences. In some examples, each cluster may contain numerous (e.g., thousands of) identical DNA sequences for sequencing or reading by a DNA sequencer. Not all of the DNA sequences may be sequenced at once, and sequencing may proceed in cycles. In some examples, the presence of a base is indicated by a release of ions, which may be detected as a change in pH, electric potential, electric current, or any other physical or chemical change. In some examples, the presence of a base is indicated by a dye, fluorescence, radiation, etc., which may be detected as emissions at characteristic wavelengths in the electromagnetic spectrum. Depending on base complexity, cluster density, or other factors, the intensity of such chemical or physical changes and of such emissions may vary. In some examples, the analyzer 114 may determine a cluster's chastity value by taking a ratio of the most intense (e.g., brightest) base in a cluster to the sum of the most intense and second most intense bases in the same cluster, each cycle, over some predetermined number of cycles, e.g., 25 cycles. A cluster may pass the chastity filter if no more than a predetermined number of base calls (e.g., 1 base call) in the cluster has a chastity value below a threshold filter amount (e.g., 60%) over the predetermined number of cycles (e.g., 25 cycles). The analyzer 114 may determine the percentage of clusters that pass the chastity filter and report such percentage as the % PF reads. In some examples, the analyzer 114 may determine chastity by a different formula, e.g., by taking a ratio of different base intensities than those described here. In some examples, the analyzer 114 may determine % PF reads using different base call, filter, and cycle amounts.

The total percentage of bases passing filter PF (% PF bases) parameter may include a percentage of bases that pass a chastity filter. As compared to the % PF reads parameter, the % PF bases parameter relates to bases instead of clusters. Thus, chastity in % PF bases may refer to the chastity of a base, rather than the chastity of a cluster. In some examples, the % PF bases parameter may be included in first result 110 or second result 112. In some examples, the chastity in % PF bases may be determined by taking a ratio of the most intense (e.g., brightest) base in a cluster or a cycle to the sum of the most intense and second most intense bases in the same cluster or cycle over some predetermined number of cycles, e.g., 25 cycles. A base call may pass the chastity filter if the base call has a chastity value above a threshold filter amount (e.g., 60%) over the predetermined number of cycles (e.g., 25 cycles). The analyzer 114 may determine the percentage of bases that pass the chastity filter and report such percentage as the % PF reads. In some examples, the analyzer 114 may determine chastity by a different formula, e.g., by taking a ratio of different base intensities than those described here. In some examples, the analyzer 114 may determine % PF reads using different base call, filter, and cycle amounts.

The total aligned reads parameter may include a number of clusters aligned to a reference sequence. In some examples, the total aligned reads parameter may be included in first result 110 or second result 112. In some examples, the analyzer 114 may count the number of clusters in the first result 110 or the second result 112 that are aligned to the reference sequence. In some examples, the analyzer 114 may report such count as the total aligned reads parameter.

The percent aligned reads parameter may include a percentage of clusters aligned to a reference sequence. In some examples, the percent aligned reads parameter may be included in first result 110 or second result 112. In some examples, the analyzer 114 may determine the percentage of clusters in the first result 110 or the second result 112 that are aligned to the reference sequence. In some examples, the analyzer 114 may report such percentage as the total aligned reads parameter.

The total aligned bases parameter may include a number of bases aligned to a reference sequence. In some examples, the total aligned bases parameter may be included in first result 110 or second result 112. In some examples, the analyzer 114 may count the number of bases in the first result 110 or the second result 112 that are aligned to the reference sequence. In some examples, the analyzer 114 may report such count as the total aligned bases parameter.

The percent aligned bases parameter may include a percentage of bases aligned to a reference sequence. In some examples, the percent aligned bases parameter may be included in first result 110 or second result 112. In some examples, the analyzer 114 may determine the percentage of bases in the first result 110 or the second result 112 that are aligned to the reference sequence. In some examples, the analyzer 114 may report such percentage as the percent aligned bases parameter.

The total probe bases parameter may include a number of bases in a DNA probe. A DNA probe may be a fragment of DNA that contains a nucleotide sequence specific to a gene or chromosome coordinate of interest. In some examples, a DNA probe may be a hybridization probe. A hybridization probe may be a fragment of DNA or RNA of variable length which can be labeled (e.g., radioactively or fluorescently) to detect the presence of nucleotide sequences that are complementary to the probe's sequence. In some examples, the total probe bases parameter may be included in first result 110 or second result 112. In some examples, the analyzer 114 may count the number of bases in the DNA probe. In some examples, the analyzer 114 may determine the start and end of a DNA probe based on characteristic markers. In some examples, such markers are characteristic DNA sequences. Thus, in some examples, the total probe bases parameter may include a count of the number of bases between a start and end marker of a DNA probe. In some examples, the total probe bases parameter may include a count of the number of bases between the start and end markers and the number of bases in the start and end markers.

The total aligned non-probe bases parameter may include a number of bases that are aligned to a reference sequence and that are not part of a probe. In some examples, the total aligned non-probe bases parameter may be included in first result 110 or second result 112. In some examples, the analyzer 114 may obtain the total aligned non-probe bases parameter by subtracting a measurement in the total probe bases parameter from a measurement in the total aligned reads parameter.

The mismatch rate parameter may include a number of mismatches between aligned sequences. In some examples, the mismatch rate parameter may be included in first result 110 or second result 112. In some examples, the mismatch rate parameter may be calculated by analyzer 114. In some examples, the analyzer 114 may compare sequences in pairwise fashion to determine if any mismatches or gaps exist between the sequences. For example, a sequence containing a variant may be compared against a reference sequence.

The amplicon mean coverage parameter may include a number obtained by dividing the total number of aligned reads to the target region by the number of targeted regions. In some examples, the mismatch rate parameter may be included in first result 110 or second result 112. In some examples, the mismatch rate parameter may be calculated by analyzer 114.

The uniformity of coverage parameter measures the spread of the reads around a mean depth of coverage and is estimated or calculated by the mean of the reads and a relevant percentile of the read distribution. In a sequencing experiment, millions of fragments corresponding to a particular part of a genome may be sequenced to produce a set of reads, which is a set of inferred sequences of base pairs corresponding to a DNA fragment. To obtain sufficient read coverage or actionable data to investigate a mutation, multiple reads may be required per sample. Ideally, each part of the genome would be read the same number of times, in which case the read coverage over the genome of interest would be uniform. Such uniformity of coverage may not be achievable in practice, thus additional sequencing may be required to achieve a desired mean read value.

The amount of such additional sequencing, and thus the uniformity of coverage, may be quantified using a fold-80 base penalty parameter. Fold-80 is based on the Picard pipeline, and refers to a multiple of additional sequencing required to ensure that 80% of the target bases achieve a mean read coverage that meets or exceeds the desired read coverage. For example, if a hundred reads generate a mean depth of coverage of 10×, then a fold-80 of 1.5 means that fifteen hundred reads would be required to generate a mean depth of coverage of 10× after factoring in the statistical distribution of the reads. Fold-80 may be calculated by dividing the mean depth of coverage by the depth of coverage at the 20^{th} percentile. A lower fold-80 score indicates high uniformity of coverage (and therefore low under- or over-sampling of the bases), while a high fold-80 score indicates a low uniformity of coverage (and therefore high under- or over-sampling of the bases). In a normal distribution, a perfectly uniformly distributed set of reads would have a fold-80 score of 1.0.

In some examples, the uniformity of coverage parameter may include a percentage of targeted base positions in which the read depth is greater than 0.2 times the mean region target coverage depth. In some examples, the uniformity of coverage parameter may be included in first result 110 or second result 112. In some examples, the uniformity of coverage parameter may be calculated by analyzer 114 based on the read depth of base regions and the mean region target coverage depth. In some examples, a different threshold value than 0.2 is used to calculate the uniformity of coverage parameter.

Thus, the analyzer 114 receives, retrieves, generates, and analyzes the analytical parameters such as those described herein from the first result 110 or second result 112. Such parameters are exemplary only, and the analyzer 114 is capable of receiving, retrieving, generating, and analyzing any additional parameters. In some examples, the analyzer 114 further receives, retrieves, generates, or analyzes additional parameters from any data generated by first DNA sequencing technique 106 or second DNA sequencing technique 108 relating to first result 110 and second result 112.

In some examples, prior to scoring by scorer 116, analyzer 114 may compare the variant localization parameters of first result 110 with second result 112 to determine whether the first result 110 and the second result 112 are directly comparable. For example, if the chromosome number, chromosome coordinate, and gene identifier of the first result 110 matches with the same of second result 112, then the analyzer 114 may determine that scoring may proceed. Any parameter may be used as a scoring gatekeeping parameter. In some examples, a flag is set to indicate whether scoring should or should not proceed with respect to a particular segment of a sequence in the first result 110 or second result 112.

In some examples, a variant may be detected in one result (e.g., first result 110) but not in another result (e.g., second result 112) at the same variant localization parameters (e.g., chromosome coordinates). To determine whether the variant reported by one of the sequencing techniques (e.g., first DNA sequencing technique 106) is a false positive, and whether the non-variant reported by another sequencing technique (e.g., second DNA sequencing technique 108) is a false negative, the analyzer 114 may provide to scorer 116 the parameters of both the variant and non-variant for scoring. Similarly, where a variant is detected in both results at the same variant localization parameters, scoring may be used to determine whether one or both reported variants are false positives. Thus, parameters corresponding to both variants may be provided to scorer 116. Scoring may equally be used to determine whether one or both non-variants reported at the same variant localization parameters are false negatives. Thus, parameters corresponding to both non-variants may also be provided to scorer 116. In some examples, to conserve system resources, scoring may be performed only when a variant is detected. In some examples, to minimize false positives and false negatives, scoring may be continuously or continually performed.

Scorer 116 may score parameters by determining a bracket to which a parameter value, V, belongs and assigning the score associated with that bracket to the parameter. For example, Table 1 below shows the brackets and scores associated with each bracket for the amplicon mean coverage parameter and the uniformity of coverage parameter:

TABLE 1
EXAMPLE PARAMETER VALUE BRACKETS AND ASSOCIATED
SCORES FOR AMPLICON MEAN COVERAGE AND
UNIFORMITY OF COVERAGE PARAMETERS

| Amplicon Mean Coverage | | Uniformity of Coverage | |
|---|---|---|---|
| (V_{A}) | Score | (Pct > 0.2 * mean) (V_{U}) | Score |
| V_{A} < 100 | 0.70 | V_{U} < 20 | 0.70 |
| 100 ≤ V_{A} < 150 | 0.80 | 20 ≤ V_{U} < 30 | 0.80 |
| 150 ≤ V_{A} < 200 | 0.90 | 30 ≤ V_{U} < 40 | 0.90 |
| 200 ≤ V_{A} < 300 | 1.00 | 40 ≤ V_{U} < 50 | 1.00 |
| 300 ≤ V_{A} < 400 | 1.10 | 50 ≤ V_{U} < 60 | 1.10 |
| 400 ≤ V_{A} < 500 | 1.20 | 60 ≤ V_{U} < 70 | 1.20 |
| 500 ≤ V_{A} < 600 | 1.30 | 70 ≤ V_{U} < 80 | 1.30 |
| V_{A} ≥ 600 | 1.40 | V_{U} ≥ 80 | 1.40 |

As an example, assume that the value for the amplicon mean coverage parameter corresponding to the sequence from the first result 110 is 47. 47 falls into the V_{A}<100 bracket. The score associated with the V_{A}<100 bracket is 0.70. Thus, scorer 116 assigns a score of 0.70 to the amplicon mean coverage parameter corresponding to the sequence from the first result 110.

As a further example, assume that the value for amplicon mean coverage parameter corresponding to the sequence from the second result 112 is 429. 429 falls into the 400≤V_{A}<500 bracket. The score associated with the V_{A}<100 bracket is 1.20. Thus, scorer 116 assigns a score of 1.20 to the amplicon mean coverage parameter corresponding to the sequence from the second result 112.

As an additional example, assume that the value for the uniformity of coverage parameter corresponding to the sequence from the first result 110 is 22.4. 22.4 falls into the 20≤V_{U}<30 bracket. The score associated with the 20≤V_{U}<30 bracket is 0.80. Thus, scorer 116 assigns a score of 0.80 to the uniformity of coverage parameter corresponding to the sequence from the first result 110.

As yet another example, assume that the value for the uniformity of coverage parameter corresponding to the sequence from the second result 112 is 88.4. 88.4 falls into the V_{U}≥80 bracket. Thus, scorer 116 assigns a score of 1.40 to the uniformity of coverage parameter corresponding to the sequence from the second result 112.

Scorer 116 may output or display the results of the scoring in any data structure. In some examples, the parameters, their corresponding values, and their corresponding scores are output or displayed as a table, such as in Table 2 below:

TABLE 2
EXAMPLE ANALYSIS AND SCORING RESULT WHERE
ONLY ONE SEQUENCING RESULT IS AVAILABLE

| | First Result 110 | | Second Result 112 | |
|---|---|---|---|---|
| Parameter | Parameter Value | Score | Parameter Value | Score |
| Sequence | AGTC | — | — | — |
| Chromosome Number | 13 | — | — | — |
| Coordinate | 13q13.1 | — | — | — |
| Gene | BRCA2 | — | — | — |
| Amplicon mean coverage | 47 | 0.70 | — | — |
| Uniformity of coverage | 22.4 | 0.80 | — | — |
| Sequence Type | Variant | — | — | — |
| rs ID Number | rs28897743 | — | — | — |
| Annotation | Pathogenic | — | — | — |
| Publication | Yes | — | — | — |
| Conclusiveness | — | Conclusive | — | — |
| | | Against | | |

As shown in Table 2, parameters are obtained for first result 110 but not for second result 112. This may occur, for example, due to peculiarities or artifacts occurring in second DNA sequencing technique 108 but not first DNA sequencing technique 106. As shown in this example, the parameters corresponding to first result 110 indicates that the sequence AGTC is a pathogenic variant. However, the conclusiveness parameter, which may be generated by the analyzer 114 or the scorer 116, indicates that the parameter values or scores are “conclusive against” the first result 110. Based on the conclusiveness parameter, a physician may conclude that the sequence AGTC as reported in first result 110 is not a true sequence. Thus, this disclosure permits the improved identification of a biological sequence to be used to detect, diagnose, prevent, treat, or otherwise manage a condition, disease, or symptom associated with the identified biological sequence.

In some examples, the conclusiveness of a result is determined by analyzing the scores generated by scorer 116. In this example, the scores for the amplicon mean coverage parameter and the uniformity of coverage parameter are 0.70 and 0.80 respectively. In some examples, the scorer 116 may be calibrated such that scores below 1.00 are indicative of untruth and scores above 1.00 are indicative of truth. In some examples, the scorer 116 may also be calibrated such that the distance from 1.00 representing the magnitude of untruth or truth. Thus, in this example, the scores of first result 110, being 0.70 and 0.80, are highly indicative of untruth.

The conclusiveness of a result may correspond with the magnitude of untruth or truth of the scores. Because numerous scores may be associated with first result 110 or second result 112, in some examples, mathematical operations may be performed to the scores to obtain a representative or overall score. For example, the representative or overall score may be a minimum, maximum, mean, median, mode, or any other representative value of the scores. In some examples, a single parameter may be selected as a representative parameter. Such singular selection may be for any reason, including the single parameter's correlation to pathogenicity, clinical diagnosis, clinical outcomes, responsiveness to treatments, etc. Thus, in some examples, a single parameter's value may be scored to provide a single representative score. Nevertheless, a plurality of parameters may also be selected as representative parameters for any reason, including the preceding reasons. Thus, in some examples, a plurality of parameters' values may be scored to provide a single representative score. Such representative or overall score may be used to determine the conclusiveness of the first result 110 or second result 112. For example, if the overall score is between 0.70 and 0.90, such score may be “conclusive against” first result 110 or second result 112. If the overall score is between 0.90 and 1.10, such score may be “inconclusive” as to first result 110 or second result 112. If the overall score is above 1.10, such score may be “conclusive in favor of” first result 110 or second result 112.

In some examples, a weighted average of the scores is used by analyzer 114 or scorer 116 to determine an overall score. The weight of each parameter may be individually determined, and the weight associated with one parameter may be different from the weight associated with another parameter. In some examples, the weight of each parameter is determined based on empirical data. In some examples, a physician may determine the weight of each parameter based on his or her experience. In some examples, the parameters are ranked in order, e.g., from the most important to least important, and weights assigned based on such ranking. In some examples, machine learning functions, such as those based on supervised learning (e.g., regression, decision tree, random forest, neural network etc.), unsupervised learning (apriori algorithm, k-means, etc.), or reinforcement learning (Markov decision process, etc.), are used to determine the weight of each parameter.

In some examples, analyzer 114 or scorer 116 uses a machine learning function to predict an output (e.g., a correct sequence) based on input parameters (e.g., those in Table 2). In some examples, analyzer 114 or scorer 116 may train the machine learning function using historical data sets containing inputs and outputs. Inputs may include a sequence (e.g., AGTC in Table 2), the sequencing technique to generate the sequence (e.g., ion semiconductor sequencing), the sequencing metrics parameters or any other parameters corresponding to the sequence, the parameters' values, the parameters' scores, etc. Outputs may include the true sequence, true sequence type (e.g., variant or reference), true annotation (e.g., pathogenic or non-pathogenic), and true clinical diagnosis (e.g., accurate clinical diagnosis of an actual patient), etc. Any other parameters, true or untrue, may be used as inputs or outputs to train the machine learning function.

In some examples, a true sequence and true sequence type may be obtained from well-established, benchmark or reference techniques known for high accuracy and reliability, such as Sanger sequencing. In some examples, true annotation and true clinical diagnosis may be obtained from medical records of actual patients whose DNA had been sequenced using such benchmark techniques. By analyzing multiple data sets, the machine learning function may learn that certain patterns of inputs result in certain patterns of outputs. Thus, if a new input such as first result 110 is fed to the machine learning function, such new input may fit a pattern associated with a particular output, e.g., a true sequence AGCC. Thus, by analyzing first result 110 using a machine learning function, analyzer 114 or scorer 116 may determine the true sequence, true sequence type, true annotation, true clinical diagnosis, or any other parameter for which the machine learning function has been trained.

In some examples, parameter scores are used as input to the machine learning function. Thus, scorer 116 may score the parameters before analysis by the machine learning function. In some examples, parameter values are used as input instead of, or in addition to, parameter scores. In other words, in some examples, scoring may not be necessary to determine a true sequence, sequence type, annotation, clinical diagnosis, etc.

In some examples, analyzer 114 or scorer 116 may compare an output of the machine learning function against a parameter associated with first result 110. For example, the sequence output by the machine learning function (e.g., AGCC) may be compared against the sequence generated by first DNA sequencing technique 106 (e.g., AGTC) or second DNA sequencing technique 108 (e.g., no result). Besides the sequence itself, any other parameters may be compared, and a user may configure analyzer 114 or scorer 116 to compare all, some, or none of the parameters. Based on a comparison of the output from the machine learning function with a corresponding input parameter (e.g., parameters associated with first result 110 or second result 112), the analyzer 114 or scorer 116 may determine the conclusiveness of the first result 110 or second result 112. For example, if an output of the machine learning function conflicts with an input parameter, the analyzer 114 or scorer 116 may determine that the output of the machine learning function is “conclusive against” the first result 110 or second result 112. If the output of the machine learning function agrees with such parameter, the analyzer 114 or scorer 116 may determine that the output of the machine learning function is “conclusive in favor of” the first result 110 or second result 112. If the output of the machine learning function neither agrees or disagrees with such parameter, the analyzer 114 or scorer 116 may determine that the output of the machine learning function is “inconclusive” with respect to the first result 110 or second result 112.

In some examples, conclusiveness may be based on the accuracy, error rate, and confidence associated with the machine learning prediction. For example, analyzer 114 or scorer 116 may determine that a machine learning output is “conclusive in favor of” or “conclusive against” an input parameter if a predetermined threshold accuracy, error rate, or confidence relating to the output of the machine learning function is met. If such predetermined threshold is not met, the result may be “inconclusive” even if the output of the machine learning function otherwise agrees or disagrees with a parameter of first result 110 or second result 112. In some examples, to reduce the number of such accuracy-related “inconclusive” results, the machine learning function may be trained with more data, validated or re-validated, changed, combined with other machine learning functions, etc., to improve the accuracy, error rate, and confidence associated with its output.

In some examples, an inconclusive result may be reached when the conclusiveness is in favor of both first result 110 and second result 112. In some examples, an inconclusive result may be reached when the conclusiveness is against both first result 110 and second result 112. In some examples, an inconclusive result may be reached when there is insufficient data to make a determination. To resolve an inconclusive result, resequencing may be performed. In some examples, resequencing using the same techniques, i.e., first DNA sequencing technique 106 or second DNA sequencing technique 108 is performed. In some examples, a third DNA sequencing technique 118 is used where the result of the analysis or scoring is inconclusive. The third DNA sequencing technique may be a well-established, benchmark or reference technique known for high accuracy and reliability, such as Sanger sequencing. Thus, in some examples, the result generated by the third DNA sequencing technique 118 may be accepted as the true result and stored as the true result in a sequencing record, patient's file, etc. In some examples, the first result 110, the second result 112, and the third result are stored and maintained in such record or file.

Table 3 shows an example of an analysis and scoring result where the sequence in first result 110 differs from the sequence in second result 112:

TABLE 3
EXAMPLE ANALYSIS AND SCORING RESULT
WHERE SEQUENCING RESULTS CONFLICT

| | First Result 110 | | Second Result 112 | |
|---|---|---|---|---|
| Parameter | Parameter Value | Score | Parameter Value | Score |
| Sequence | AGTC | — | AGCC | — |
| Chromosome Number | 13 | — | 13 | — |
| Coordinate | 13q13.1 | — | 13q13.1 | — |
| Gene | BRCA2 | — | BRCA2 | — |
| Amplicon mean coverage | 47 | 0.70 | 429 | 1.20 |
| Uniformity of coverage | 22.4 | 0.80 | 88.4 | 1.40 |
| Sequence Type | Variant | — | Variant | — |
| rs ID Number | rs28897743 | — | rs28897743 | — |
| Annotation | Pathogenic | — | Pathogenic | — |
| Publication | Yes | — | Yes | — |
| Conclusiveness | — | Conclusive | — | Conclusive |
| | | Against | | in Favor of |

In this example, first result 110 indicates that the sequence is AGTC, while the second result 112 indicates that the sequence is AGCC. Without the benefit of the analysis, scoring, or conclusiveness disclosed herein, a physician faced with such conflicting sequencing results may reject both first result 110 and second result 112. In some examples, to resolve such conflicting results, the physician may perform a resequencing using first DNA sequencing technique 106 and second DNA sequencing technique 108 to verify first result 110 and second result 112. In some examples, the physician may use a different DNA sequencing technique, such as third DNA sequencing technique 118 to obtain a third result.

However, on the basis of parameter values and scores provided by analyzer 114 and scorer 116, a physician may also determine that the first result 110 is most likely untrue, and the second result 112 is most likely true. As earlier explained, the likelihood of untruth increases below a score of 1.00, and the likelihood of truth increases above a score of 1.00. Here, the scores of 0.70 and 0.80 are significantly below 1.00, thus the likelihood of untruth is high with respect to first result 110. On the other hand, the scores of 1.20 and 1.40 are significant above 1.00, thus the likelihood of truth is high with respect to second result 112. At this point, a physician may conclude that second result 112 (AGTC) is true, while first result 110 (AGCC) is untrue.

Additionally, the physician may also look to the conclusiveness score to guide his or her determination as to the true result. As explained above, the conclusiveness score may be based on a weighted average of the scores and/or an analysis of the input parameters (e.g., the parameters corresponding to first result 110 and second result 112) using trained machine learning functions. Further, the output of such machine learning functions may be validated and subject to a minimum accuracy threshold. In other words, the conclusiveness score is a more robust indicator of the truth or untruth of first result 110 and second result 112, for example, as compared to a physician's “gut feel” from glancing over parameter values.

In Table 3, the conclusiveness score indicates that the analysis and/or scoring performed by the analyzer 114 and/or scorer 116 is “conclusive against” the first result 110, but “conclusive in favor of” the second result 112. Thus, based on the conclusiveness score, the physician may determine that AGTC in the second result 112 is true, while AGCC in the first result 110 is untrue. In some examples, analyzer 114 or scorer 116 determines the true sequence among the conflicting results based on the conclusiveness score, and may output or display such true sequence together with the parameters and scores of the analysis and scoring. For example, if the first result 110 is determined to be true based on the overall score or the conclusiveness score, then analyzer 114 or scorer 116 may likewise determine that the sequence in the first result 110 is true. Analyzer 114 or score 116 may indicate that the first result is true using some indication, such as a flag, marking, sound, notification, display, or other output that indicates first result 110 or any parameter in the first result 110, such as the sequence (e.g., AGTC), sequence type, annotation, etc., is true. First result 110 with such indication of truth may be referred to as first result (true), 110T. Likewise, if the second result 112 is determined to be true based on the conclusiveness score, then second result 112 with such indication of truth may be referred to as second result (true), 112T.

In some examples, the conclusiveness scores of both the first result 110 and the second result 112 are analyzed as a tuple. For example, first result 110 may only be determined to be true if the combination of the conclusiveness scores of both the first result 110 and the second result 112 support such a determination. If the combination of the conclusiveness scores of the first result 110 is “conclusive in favor of” and that of the second result 112 is “conclusive against,” then the analyzer 114 or scorer 116 may determine that the first result 110 is true and further indicate that first result 110 is true, thereby generating first result (true) 110T. But if the combination of the conclusiveness scores of the second result 112 is “conclusive against” first result 110 and “conclusive in favor of” second result 112, then the analyzer 114 or scorer 116 may determine that the second result 112 is true and further indicate that the second result 112 is true, thereby generating second result (true) 112T.

In some examples, if the combination of the conclusiveness scores of the first result 110 is “conclusive in favor of” but no conclusiveness score is received for second result 112, then the analyzer 114 or scorer 116 may determine that the first result 110 is true and further indicate that the first result 110 is true, thereby generating first result (true) 110T. But if the combination of the conclusiveness scores of the second result 112 is “conclusive in favor of” but no conclusiveness score is received for first result 110, then the analyzer 114 or scorer 116 may determine that the second result 112 is true and indicate that the second result 112 is true, thereby generating second result (true) 112T.

In some examples, if the combination of the conclusiveness scores of the first result 110 is “conclusive in favor of” and that of the second result 112 is also “conclusive in favor of,” then the analyzer 114 or scorer 116 may determine that the conclusiveness scores conflict. Analyzer 114 or scorer 116 may then output or display “inconclusive” instead of “conclusive in favor of” as the conclusiveness scores for each of first result 110 and second result 112. Similarly, if the combination of the conclusiveness scores of the first result 110 is “conclusive against” and that of the second result 112 is also “conclusive against,” then the analyzer 114 or scorer 116 may determine that the conclusiveness scores conflict. Analyzer 114 or scorer 116 may then output or display “inconclusive” instead of “conclusive against” as the conclusiveness scores for each of first result 110 and second result 112.

Table 4 shows an example of an analysis and scoring result where the sequence in first result 110 is the same as the sequence in second result 112, but the results are contradicted by the conclusiveness scores:

TABLE 4
EXAMPLE ANALYSIS AND SCORING RESULT WHERE
SEQUENCING RESULTS ARE CONSISTENT, BUT CONCLUSIVENESS
SCORES CONTRADICTS THE RESULTS

| | First Result 110 | | Second Result 112 | |
|---|---|---|---|---|
| Parameter | Parameter Value | Score | Parameter Value | Score |
| Sequence | AGCC | — | AGCC | — |
| Chromosome Number | 13 | — | 13 | — |
| Coordinate | 13q13.1 | — | 13q13.1 | — |
| Gene | BRCA2 | — | BRCA2 | — |
| Amplicon mean coverage | 47 | 0.70 | 429 | 1.20 |
| Uniformity of coverage | 22.4 | 0.80 | 88.4 | 1.40 |
| Sequence Type | Variant | — | Variant | — |
| rs ID Number | rs28897743 | — | rs28897743 | — |
| Annotation | Pathogenic | — | Pathogenic | — |
| Publication | Yes | — | Yes | — |
| Conclusiveness | — | Conclusive | — | Conclusive |
| | | Against | | in Favor of |

In this example, both first result 110 and second result 112 indicate that the sequence is AGCC, however, the conclusiveness scores corresponding to first result 110 and second result 112 contradict each other. Specifically, the conclusiveness score corresponding to first result 110 indicates that the parameter values and/or scores in first result 110 are “conclusive against” the first result 110, suggesting that the sequence AGCC in first result 110 is untrue. At the same time, the conclusiveness score corresponding to second result 112 indicates that the parameter values and/or scores in first result 112 are “conclusive in favor of” the second result 112, suggesting that the sequence AGCC in first result 110 is true. Thus, according to the conclusiveness score, AGCC is at once true and untrue, resulting in a contradiction.

Without the benefit of the parameter values, parameter scores, or conclusiveness score, a physician may conclude based on the agreement of first result 110 with second result 112 that the sequence in both results (AGCC) is true. However, this may be a false positive, as indicated by the contradictory parameter scores and conclusiveness scores.

Thus, when the analyzer 114 or scorer 116 detects such a contradiction, analyzer 114 or scorer 116 may provide a recommendation or an instruction to a user or a DNA sequencing system to perform a resequencing using the same sequencing techniques (e.g., first DNA sequencing technique 106 and second DNA sequencing technique 108) or using a different sequencing technique or techniques (e.g., third DNA sequencing technique 118). Providing may be displaying, outputting, sending, transmitting, broadcasting, generating, creating, making, etc. A recommendation may be a notification, suggestion, advice, counsel or any other non-imperative communication in any form. An instruction may be an imperative communication in human-comprehensible, machine-readable, or any form. In some examples, the recommendation or instruction may be included in the analysis and scoring results, for example, as one of the parameters or in a separate dialog box. In some examples, a recommendation may influence or cause a physician to perform a resequencing. In some examples, an instruction may cause a DNA sequencing system to perform a resequencing.

The third DNA sequencing technique may be a well-established, benchmark or reference technique known for high accuracy and reliability, such as Sanger sequencing. In some examples, the result generated by the third DNA sequencing technique (i.e., third result) will agree with the first result 110 or second result 112. In some examples, the result generated by the third DNA sequencing technique will agree with the first result 110 and the second result 112 (such as in the example in Table 4, where the sequence in first result 110 and the second result 112 are the same). In some examples, the third result will not agree with either the first result 110 or the second result 112.

To illustrate how false positives and false negatives may be prevented, assume that the recommended or instructed resequencing reveals that the first result 110 and the second result 112 are both wrong. For example, the third result may reveal that the true sequence is AGTC, which is different from AGCC (the sequence in first result 110 and second result 112). Assuming AGTC is not pathogenic, while AGCC is pathogenic, then the recommendation or instruction made by the analyzer 114 or scorer 116 would have averted an otherwise convincing false positive (since both first result 110 and second result 112 agree). On the other hand, assuming AGTC is pathogenic, while AGCC is not pathogenic, then the recommendation or instruction made by the analyzer 114 or scorer 116 would have averted an otherwise equally convincing false negative.

FIG. 2 is a flow diagram illustrating a method for analyzing and scoring results from a plurality of DNA sequencing techniques, and determining whether a sequencing result is true, in accordance with various examples of the present disclosure. The method 200 may be performed by non-transitory memory and processors provided, for example, by the system 100 described with respect to FIG. 1. Additional steps may be provided before, during, and after the steps of method 200, and some of the steps described may be replaced, eliminated and/or re-ordered for other examples of the method 200. Method 200 may also include additional steps and elements, such as those described with respect to FIG. 1. In some examples, method 200 may be performed by one or more computer systems 100 described with respect to FIG. 1, acting in concert such as in a distributed or cloud computing network or individually such as on a single server or workstation.

At action 202, a first DNA sequencing result and a second DNA sequencing result is received. In some examples, the first DNA sequencing result is a result of a first DNA sequencing technique, and the second DNA sequencing result is a result of a second DNA sequencing technique. A DNA sequencing result may be a result of a DNA sequencing technique when a DNA sequencing technique is used to sequence at least some part of that DNA sequencing result. In some examples, a DNA sequencing result may include unaligned DNA fragments comprising one or more nucleotides. In some examples, a DNA sequencing result may include aligned DNA fragments comprising one or more nucleotides. In some examples, the sequence of the aligned fragments may correspond to the sequence of an allele or a gene.

In some examples, the first DNA sequencing result and the second DNA sequencing result is received over a network, such as the internet or a local area network, or over a direct wireless communication such as Bluetooth and near-field communication (NFC). In some examples, the first DNA sequencing result and the second DNA sequencing result is received over a system or other bus or circuit board interconnects.

At action 204, a difference is determined between the first DNA sequencing result and the second DNA sequencing result. In some examples, a difference between a first DNA sequence in the first DNA sequencing result and a second DNA sequence in the second DNA sequencing result is determined. For instance, as shown in Table 3 described with respect to FIG. 1, a difference between first DNA sequence (AGTC) and second sequence DNA sequence (AGCC) is that the third nucleotide in the first DNA sequence, thymine (T), differs from the third nucleotide in the second DNA sequence, cytosine (C). In some examples, determining a difference between the first DNA sequencing result and the second DNA sequencing result includes merely determining that a difference exists between the first DNA sequencing result and the second DNA sequencing result, without identifying what that difference is. For example, determining that AGTC is different from AGCC, without identifying that a particular nucleotide at a particular position in a first DNA sequencing result is different from a nucleotide at a corresponding position in a second DNA sequencing result.

In some examples, determining a difference between the first DNA sequencing result and the second DNA sequencing result includes determining a difference in parameter values between the first DNA sequencing result and the second DNA sequencing result. For instance, as shown in Table 3 described with respect to FIG. 1, a difference between the amplicon mean coverage parameter of the first result 110 and the amplicon mean coverage parameter of the second result 112 may be determined. In some examples, a difference is determined by subtracting one value from the other, and if the result is not 0, then a difference exists. With reference to Table 3, the amplicon mean coverage value of the first result 110, which is 47, may be subtracted from the amplicon mean coverage value of the second result 112, which is 429. The result is 382, which is not 0. Thus, it may be determined that a difference exists between the first result 110 and the second result 112. In some examples, determining a difference between the first DNA sequencing result and the second DNA sequencing result includes merely determining that a difference exists between the first DNA sequencing result and the second DNA sequencing result, without identifying what that difference is. For example, determining that 429 is different from 47, without identifying or calculating the mathematical difference between 429 and 47.

In some examples, determining a difference includes comparing numbers, alphanumeric characters, text strings, or any other type of data in a parameter value, such as chromosome numbers (e.g., “13q13.1” vs. “13q12.3”), annotations (e.g., “pathogenic” vs. “benign”), sequence types (e.g., “Variant” vs. “Reference”), rs ID numbers (e.g., “rs28897743” vs “r51799944”), etc.

At action 206, parameters corresponding to the first DNA sequencing result and to the second DNA sequencing result are scored. In some examples, parameters corresponding to the first DNA sequencing result and to the second DNA sequencing result include parameters that are common between the first DNA sequencing result and the second DNA sequencing result, i.e., the union of parameters of the first DNA sequencing result and the second DNA sequencing result. In some examples, such parameters that are at the union of the parameters of the first DNA sequencing result and the second DNA sequencing result are scored.

In some examples, the parameters of each sequencing result are scored independent of the relationship (e.g., a union) between the parameters of each sequencing result. Thus, some or all of the parameters corresponding to the first DNA sequencing result may be scored to generate a first scored set of parameters, and some or all of the parameters corresponding to the second DNA sequencing result may be scored to generate a second scored set of parameters. Thus, if any parameters are included in the first sequencing result but not the second sequencing result, such additional parameters may be scored as well. Under this scoring regime, the parameters that are not common between the first DNA sequencing result and the second DNA sequencing result may still be scored.

At action 208, a value range of a parameter in a set of reference parameters corresponding to a value of a corresponding parameter of the parameters is determined. For example, Table 1 described with respect to FIG. 1 shows a number of value ranges of the amplicon mean coverage parameter, A: V_{A}<100; 100≤V_{A}<150; 150≤V_{A}<200; and so forth, where V denotes a value of a parameter in general and V_{A} denotes the value of the amplicon mean coverage parameter. Thus, V_{A}<100 may be referred to as a first amplicon mean coverage parameter value range; 100≤V_{A}<150 may be referred to as a second amplicon mean coverage parameter value range; and 150≤V_{A}<200 may be referred to as a third amplicon mean coverage parameter value range.

The value of the amplicon mean coverage parameter analyzed or received from a DNA sequencing result may be any value. In the example shown in Table 2 described with respect to FIG. 1, the value of the amplicon mean coverage parameter analyzed or received from first result 110 is 47. The corresponding value range for an amplicon mean coverage parameter value of 47 is V_{A}<100. Such value range lookups may be performed for any parameter analyzed or received from the DNA sequencing result against any corresponding parameters associated with a value range, such as the amplicon mean coverage example above. Such parameters associated with a value range may be referred to as reference parameters. A plurality of such parameters may be referred to as a set of reference parameters.

At action 210, a score associated with the value range of the parameter in the set of reference parameters is assigned as a score of the corresponding parameter. For example, Table 2 shows value ranges and their associated scores. Specifically, the value range V_{A}<100 is associated with a score of 0.70; the value range 100≤V_{A}<150 is associated with a score of 0.80; the value range 150≤V_{A}<200 is associated with a score of 0.90; and so forth. Thus, the score associated with an amplicon mean coverage parameter value of 47, which falls under the V_{A}<100 value range, is 0.70.

At action 212, the first DNA sequencing result or the second DNA sequencing result is determined as conclusive or inconclusive based on respective scores of the parameters of the first DNA sequencing result and the second DNA sequencing result. In some examples, a weighted average of the parameter scores corresponding to a DNA sequencing result may be taken to obtain an overall or representative score. In some examples, other mathematical operations are performed to obtain an overall or representative score. In some examples, no mathematical operations are performed to obtain an overall or representative score, such as when a single parameter's score is taken as representative. In some examples, the overall or representative scores correspond to a DNA sequence at a specific coordinate known to be pathogenic or likely to be pathogenic. If the overall score is below a threshold value, such as 1.0, then the DNA sequencing result may be determined to be inconclusive. However, if the overall score is above a threshold value, such as 1.0, then the DNA sequencing result may be determined to be conclusive. In some examples, if output from a machine learning function as to the value of a parameter (e.g., a sequence) is inconsistent with a corresponding parameter (e.g., a sequence) in the DNA sequencing result, the DNA sequencing result may be determined to be inconclusive.

At action 214, an indication to perform a third DNA sequencing using a third DNA sequencing technique is provided when the first DNA sequencing result or the second DNA sequencing result is inconclusive. In some examples, an indication to perform a third DNA sequencing using a third DNA sequencing technique is provided when the tuple of the conclusive scores corresponding to the first DNA sequencing result and to the second DNA sequencing result is inconclusive. In some examples, if one of the DNA sequencing results is conclusive but the other DNA sequencing result is inconclusive, the tuple is inconclusive. In some examples, if one sequencing result is highly conclusive but the other sequencing result is inconclusive, the tuple is conclusive.

### Comprehensive Hereditary Cancer Testing

To date, at least 36 genes have been linked to an increased risk for human cancer. Genetic testing results indicating an increased risk for one or more cancers may inform a patient's family members, who may be at higher risk as well. With the knowledge of a higher risk of cancer, patients and their physicians can put together a screening plan at a younger age. In more serious cases, the result may indicate a very high risk of cancer in the future. If appropriate, surgical intervention may be suggested to prevent cancer. Exemplary clinical features that have been linked to genetic mutations include, but are not limited to, Astrocytoma, Colon cancer, Carious teeth, Epidermoid cysts, Keloids, Medulloblastoma, Odontoma, Osteoma, Increased number of teeth, Unerupted tooth, Desmoid tumors, Hyperpigmentation of the skin, Fibroadenoma of the breast, Hepatoblastoma, Adrenocortical adenoma, Adrenocortical carcinoma, Gastric polyposis, Papillary thyroid carcinoma, Duodenal adenocarcinoma, Congenital hypertrophy of retinal pigment epithelium, Duodenal polyposis, Multiple lipomas, Adenomatous colonic polyposis, and Small intestine carcinoid.

The following genes are known to be linked to increased risk for at least eight human cancers:

| GENE | CANCER(S) |
|---|---|
| APC | Colorectal, Gastric, Pancreatic |
| ATM | Breast, Pancreatic, Prostate |
| BARD1 | Breast |
| BMPR1A | Colorectal, Gastric, Pancreatic |
| BRCA1 | Breast, Ovarian, Melanoma, Pancreatic, Prostate |
| BRCA2 | Breast, Ovarian, Melanoma, Pancreatic, Prostate |
| BRIP1 | Breast, Ovarian, Prostate |
| CDH1 | Breast, Colorectal, Gastric |
| CDK4 | Melanoma, Pancreatic |
| CDKN2A | Melanoma, Pancreatic |
| CHEK2 | Breast, Colorectal, Prostate |
| ELAC2 | Prostate |
| EPCAM | Colorectal, Endometrial, Gastric, Ovarian, Pancreatic, Prostate |
| HOXB13 | Prostate |
| MLH1 | Colorectal, Endometrial, Gastric, Ovarian, Pancreatic, Prostate |
| MRE11A | Breast, Ovarian, Prostate |
| MSH2 | Colorectal, Endometrial, Gastric, Ovarian, Pancreatic, Prostate |
| MSH6 | Colorectal, Endometrial, Gastric, Ovarian, Pancreatic, Prostate |
| MUTYH | Colorectal |
| NBN | Breast, Prostate |
| PALB2 | Breast, Pancreatic, Prostate |
| PMS2 | Colorectal, Endometrial, Gastric, Ovarian, Pancreatic, Prostate |
| POLD1 | Colorectal |
| POLE | Colorectal |
| PTCH1 | Breast, Colorectal |
| PTEN | Breast, Colorectal, Endometrial, Melanoma |
| RAD50 | Breast |
| RAD51C | Breast, Ovarian, Prostate |
| RAD51D | Ovarian, Prostate |
| RECQL4 | Breast |
| RET | Ovarian |
| RINT1 | Breast |
| SMAD4 | Colorectal, Gastric, Pancreatic |
| STK11 | Breast, Colorectal, Endometrial, Gastric, Ovarian, Pancreatic |
| TP53 | Breast, Colorectal, Endometrial, Gastric, Melanoma, Ovarian, |
| | Pancreatic, Prostate |
| NF1 | Breast |

### Comprehensive Non-Invasive Carrier Screening

In other embodiments, next generation sequencing techniques are useful in non-invasive carrier screening methods for detecting gene mutations (defects in the genes) that are linked to many of the most common and devastating diseases that can be passed unknowingly from parent to child. Newborn screening is an example of one such test for parents and prospective parents that provides information on whether an individual has gene mutation(s) that are linked to a specific inheritable disease.

The results of such genetic sequencing provide information on the likelihood that a child will inherit one of a number of rare diseases. Because many genetic diseases are recessive, it is possible for someone to have 1 copy of the faulty gene without having any symptoms. This can go on for many generations without anyone recognizing that there is a mutation being passed down. These individuals are called “carriers” because they carry a faulty gene. The concern is that if an individual is a carrier and they want to have children with someone who is also a carrier, there is a risk that their children will wind up with 2 copies of the mutation and inherit the disease.

Exemplary genes (and their associated genetic disorder) that may be analyzed in accordance with the methods disclosed herein include, without limitation,
ACTA2 Familial Thoracic Aortic Aneurysm 6
AKAP9 Long QT syndrome 11
ASPA Familial Canavan Disease
BCKDHA Maple Syrup Urine Disease, Type 1A
BCKDHB Maple Syrup Urine Disease, Type 1B
BLM Bloom Syndrome
CA12 Hyperchlorohidrosis
CDH23 Usher Syndrome, Type 1D
CFTR Cystic Fibrosis
COL11A2 Deafness, Autosomal dominant 13
COL3A1 Ehlers-Danlos Syndrome, Type 4
COL4A1 Hereditary angiopathy, w/ nephropathy, aneurysm, and muscle cramps
DBT Maple Syrup Urine Disease, Type 2
DLD Maple Syrup Urine Disease, Type 3
DYNC1H1 Spinal muscular atrophy, lower extremity 1, autosomal dominant
ELP1 Familial Dysautonomia
FANCA Fanconi anemia, complementation group A
FANCC Fanconi anemia, complementation group C
FANCF Fanconi anemia, complementation group F
FANCG Fanconi anemia, complementation group G
FBN1 Marian Syndrome
FMR1 Fragile X Syndrome
GAA Glycogen Storage Disease, Type II
GALT Galactosemia
GBA Gaucher Disease
GBE1 Glycogen Storage Disease, Type IV
GJB2 Deafness, autosomal dominant 3a
GJB3 Deafness, autosomal dominant 2b
GJB6 Deafness, autosomal dominant 3b
HBA1 α-Thalassemia
HBA2 α-Thalassemia
HBB β-Thalassemia
HEXA Tay-Sachs disease
KCNE1 Long QT syndrome 5
KCNE2 Long QT syndrome 6
KCNQ1 Jervell and Lange-Nielsen Syndrome 1
KCNQ4 DFNA 2 Nonsyndromic Hearing Loss
MCOLN1 Mucolipidosis, Type IV
MYH11 Aortic Aneurysm, familial thoracic 4
MYLK Aortic aneurysm, familial thoracic 7
MYO7A Usher Syndrome, Type 1A, Type 1, Type 1B
NPC1 Niemann-Pick Disease, Type C1
NPC2 Niemann-Pick Disease, Type C2
OTC Ornithine carbamoyltransferase deficiency
PAH Phenylketonuria
PCDH15 Usher Syndrome, Type 1F
SCN5A Long QT Syndrome 3
SCNN1A Liddle Syndrome 3
SCNN1B Pseudoprimary Hyperaldosteronism
SCNN1G Liddle Syndrome 2
SLC26A4 Pendred Syndrome
SLC2A10 Arterial Tortuosity Syndrome
SMAD3 Loeys-Dietz Syndrome 3
SMN1 Werdnig-Hoffmann Disease
SMN2 Spinal Muscular Atrophy
TGFBR1 Loeys-Dietz Syndrome 1
TGFBR2 Loeys-Dietz Syndrome 2
UBA1 Spinal Muscular Atrophy, X-Linked 2
USH1C Usher Syndrome, Type 1C
USH2A Usher Syndrome, Type 2A
VAPB Adult Proximal Spinal Muscular Atrophy

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

The term “adapter,” as used herein, refers to unique sequences used to cap the ends of a fragmented DNA. The adapter's functions are as follows: 1) allow hybridization to solid surface; 2) provide priming location for both amplification and sequencing primers; and 3) provide barcoding for multiplexing different samples in the same run.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

“Coverage,” as used herein refers to the number of times a particular nucleotide is sequenced. Due to the error-prone sequencing reactions, random errors could occur. Therefore, 30× coverage is typically required to ensure each nucleotide sequence is accurate.

“Deep Sequencing,” as used herein refers to sequencing where the coverage is greater than 30×. This is used in cases where dealing with rare polymorphisms which only a subset of the sample expresses the mutation. This method increases range, complexity, sensitivity, and accuracy of the result.

The term “for example” or “e.g.,” as used herein, is used merely by way of example, without limitation intended, and should not be construed as referring only those items explicitly enumerated in the specification.

A “homopolymer,” as defined herein, is a stretch of single nucleotide bases, such as AAAA or GGGGGG.

As used herein, the term “library” refers to a collection of DNA fragments with adapters ligated to each end. Library preparation is required before a sequencing run.

“Mate-Paired Reads,” refers to a sample preparation step where large DNA fragments (˜10 kb) are circularized with an adapter sequence followed by degradation of the circular DNA. This method links DNA fragments that are separated from each other by a certain distance and it is used in applications such as de novo assembly, structural variant detection, and identification of complex genomic rearrangements.

“Next Generation Sequencing”, or NGS, as used herein is meant to encompass a sequencing method where millions of sequencing reactions are carried out in parallel, increasing the sequencing throughput.

“Paired-End Sequencing,” as used herein refers to the process of sequencing from both ends of a fragment while keeping track of the paired data. With this method the sequencing reaction will commence from one end of the fragment. Once completed, the fragment is denatured and a sequencing primer is hybridized to the reverse side adapter. The fragment is then sequenced again. Using this method will allow either further confirmation of the accuracy of the sequence or it could be used to increase the overall read length.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

“Read Length,” as used herein refers to the length of each sequencing read. This variable is always represented as an average read length since individual reads have varying lengths.

“Reads,” as used herein refers to the output of an NGS sequencing reaction. A read is a single uninterrupted series of nucleotides representing the sequence of the template.

As used herein, “Reference sequence” or “reference genome” refers to a fully sequenced and mapped genome used for the mapping of sequence reads.

“Specificity, as used herein, refers to the percentage of sequences that map to the intended targets out of total bases per run.

As used herein, “synthetic” shall mean that the material is not of a human or animal origin.

“Uniformity, as used herein, refers to the variability in sequence coverage across target regions. When performing whole genome sequencing or exome sequencing, it is expected that the result will be highly uniform (as there should be a 1:1 ratio in the starting material). However, RNA sequencing will not be uniform since differences in expression alter its starting material.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### REFERENCES

The following references, to the extent that they provide exemplary procedural or other details supplementary to those set forth herein, are specifically incorporated herein in their entirety by express reference thereto:
ADZHUBEI, I et al., “A method and server for predicting damaging missense mutations,” Nature Methods, 7(4):248-49 (2010)
ALBERTS, B, MOLECULAR BIOLOGY OF THE CELL, 5^{th} Edition, Garland Science, New York (2008).
ALTSCHUL, S F et al., “Gapped BLAST and PSI-BLAST: a new generation of protein database search programs,” Nucl. Acids Res., 25(17):3389-3402 (September 1997).
EID, J, et al., “Real-time DNA sequencing from single polymerase molecules,” Science, 323(5910): 133-138 (2009).
EWING, B et al., “Base-calling of automated sequencer traces using Phred,” Genome Res., 8(3):175-194 (1998).
GRIBSKOV, M and BURGESS, R R, “Sigma factors from E. coli, B. subtilis, phage SP01, and phage T4 are homologous proteins,” Nucleic Acids Res., 14(16):6745-6763 (August 1986).
HALE, W G, and MARGHAM, J P, “HARPER COLLINS DICTIONARY OF BIOLOGY,” HarperPerennial, New York (1991).
HARDMAN, J G, and LIMBIRD, L E, (Eds.), “GOODMAN AND GILMAN'S THE PHARMACOLOGICAL BASIS OF THERAPEUTICS” 10^{th} Edition, McGraw-Hill, New York (2001).
KYTE, J and DOOLITTLE, R F “A simple method for displaying the hydropathic character of a protein,” J. Mol. Biol., 157(1):105-132 (May 1982).
METZKER, M L, “Emerging technologies in DNA sequencing,” Genome Res., 15(12):1767-1776 (2005).
NEEDLEMAN, S B and WUNSCH, C D, “A general method applicable to the search for similarities in the amino acid sequence of two proteins,” J. Mol. Biol., 48(3):443-453 (March 1970).
PENNISI, E, “Semiconductors inspire new sequencing technologies,” Science, 327(5970):1190 (2010).
SCHRODINGER 2013. Schrodinger, LLC: New York, N.Y. (2013).
SCHWARZ, J et al., “MutationTaster2: mutation prediction for the deep-sequencing age,” Nature Meth., 11(4):361-362 (2014)
SIM, N-L et al., “SIFT web server: predicting effects of amino acid substitutions on proteins,” Nucleic Acids Res., 40(1):452-457 (2012).
SINGLETON, P and SAINSBURY, D, “DICTIONARY OF MICROBIOLOGY AND MOLECULAR BIOLOGY,” 2^{nd} Ed., John Wiley and Sons, New York (1987).
VASER, R et al., “SIFT missense predictions for genomes,” Nature Protocols, 11:1-9 (2016).

In the foregoing description, numerous details are set forth. It will be apparent, however, to one of ordinary skill in the art having the benefit of this disclosure, that the present disclosure may be practiced without these specific details. For example, the present disclosure may apply to any number of sequencing techniques or results, and are not limited specifically to DNA sequencing techniques or results, or to just two sequencing techniques or to two results. For example, RNA sequencing techniques, amino acid sequencing techniques, protein sequencing techniques, or any other types of chemical or biological sequencing techniques are also contemplated. In some instances, well-known structures and devices are shown in block diagram form, rather than in detail, in order to avoid obscuring the present disclosure. Although illustrative examples have been shown and described, a wide range of modification, change and substitution is contemplated in the foregoing disclosure and in some instances, some features of the examples may be employed without a corresponding use of other features. In some instances, actions may be performed according to alternative orderings. One of ordinary skill in the art would recognize many variations, alternatives, and modifications. Thus, the scope of the invention should be limited only by the following claims, and it is appropriate that the claims be construed broadly and in a manner consistent with the scope of the examples disclosed herein.

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

All such similar substitutes and modifications apparent to those skilled in the art are deemed to be within the spirit, scope and concept of the invention as defined by the appended claims.

What is claimed is:

1. A system comprising:
a non-transitory memory; and
one or more hardware processors coupled to the non-transitory memory to execute instructions from the non-transitory memory to perform operations comprising:
receiving a first DNA sequencing result and a second DNA sequencing result, wherein the first DNA sequencing result is a result of a first DNA sequencing technique and the second DNA sequencing result is a result of a second DNA sequencing technique;
determining a difference between the first DNA sequencing result and the second DNA sequencing result;
scoring parameters corresponding to the first DNA sequencing result and to the second DNA sequencing result, wherein the scoring includes:
determining a value range of a parameter in a set of reference parameters corresponding to a value of a corresponding parameter of the parameters; and
assigning a score associated with the value range of the parameter in the set of reference parameters as a score of the corresponding parameter;
determining that the first DNA sequencing result or the second DNA sequencing result is conclusive or inconclusive based on respective scores of the parameters of the first DNA sequencing result and the second DNA sequencing result; and
indicating to perform a third DNA sequencing using a third DNA sequencing technique when the first DNA sequencing result or the second DNA sequencing result is inconclusive.

2. The system of claim 1, further comprising:
indicating to perform the third DNA sequencing when the first DNA sequencing result and the second DNA sequencing result, taken together, are inconclusive.

3. The system of claim 1, wherein the first and the second DNA sequence techniques are performed in automated, massively parallel, fashion.

4. The system of claim 1, wherein the first DNA sequencing technique includes an ion semiconductor-based detection system.

5. The system of claim 1, wherein the second DNA sequencing technique includes one or more of tagmentation, reduced-cycle amplification, bridge amplification, clonal amplification, and reversible dye-termination.

6. A non-transitory machine-readable medium having stored thereon machine-readable instructions executable to cause at least one machine to perform operations comprising:
receiving a first DNA sequencing result and a second DNA sequencing result, wherein the first DNA sequencing result is a result of a first DNA sequencing technique and the second DNA sequencing result is a result of a second DNA sequencing technique;
determining a difference between the first DNA sequencing result and the second DNA sequencing result;
scoring parameters corresponding to the first DNA sequencing result and to the second DNA sequencing result, wherein the scoring includes:
determining a value range of a parameter in a set of reference parameters corresponding to a value of a corresponding parameter of the parameters; and
assigning a score associated with the value range of the parameter in the set of reference parameters as a score of the corresponding parameter;
determining that the first DNA sequencing result or the second DNA sequencing result is conclusive or inconclusive based on respective scores of the parameters of the first DNA sequencing result and the second DNA sequencing result; and
indicating to perform a third DNA sequencing using a third DNA sequencing technique when the first DNA sequencing result or the second DNA sequencing result is inconclusive.

7. The non-transitory machine-readable medium of claim 6, further comprising:
indicating to perform the third DNA sequencing when the first DNA sequencing result and the second DNA sequencing result, taken together, are inconclusive.

8. The non-transitory machine-readable medium of claim 6, wherein the first and the second DNA sequence techniques are performed in automated, massively parallel, fashion.

9. The non-transitory machine-readable medium of claim 6, wherein the first DNA sequencing technique includes an ion semiconductor-based detection system.

10. The non-transitory machine-readable medium of claim 6, wherein the second DNA sequencing technique includes one or more of tagmentation, reduced-cycle amplification, bridge amplification, clonal amplification, and reversible dye-termination.

11. A method of increasing the accuracy, or improving the fidelity of, a high-throughput DNA sequencing process, comprising:
receiving a first DNA sequencing result and a second DNA sequencing result, wherein the first DNA sequencing result is a result of a first DNA sequencing technique and the second DNA sequencing result is a result of a second DNA sequencing technique;
determining a difference between the first DNA sequencing result and the second DNA sequencing result;
scoring parameters corresponding to the first DNA sequencing result and to the second DNA sequencing result, wherein the scoring includes:
determining a value range of a parameter in a set of reference parameters corresponding to a value of a corresponding parameter of the parameters; and
assigning a score associated with the value range of the parameter in the set of reference parameters as a score of the corresponding parameter;
determining that the first DNA sequencing result or the second DNA sequencing result is conclusive or inconclusive based on respective scores of the parameters of the first DNA sequencing result and the second DNA sequencing result; and
indicating to perform a third DNA sequencing using a third DNA sequencing technique when the first DNA sequencing result or the second DNA sequencing result is inconclusive.

12. The method of claim 11, further comprising:
indicating to perform the third DNA sequencing when the first DNA sequencing result and the second DNA sequencing result, taken together, are inconclusive.

13. The method of claim 11, wherein the first and the second DNA sequence techniques are performed in automated, massively parallel, fashion.

14. The method of claim 11, wherein the first DNA sequencing technique includes an ion semiconductor-based detection system.

15. The method of claim 11, wherein the second DNA sequencing technique includes one or more of tagmentation, reduced-cycle amplification, bridge amplification, clonal amplification, and reversible dye-termination.

16. The method of claim 11, wherein the set of reference parameters include at least one parameter selected from the group consisting of: allele frequency, read quality index, mean coverage, and uniformity of coverage.

17. The method of claim 11, wherein a first knowledge score is used to express a degree of knowledge of the first DNA sequencing result in medical literature or a clinical database and a second knowledge score is used to express a degree of knowledge of the second DNA sequencing result in medical literature or a clinical database.

18. The method of claim 16, wherein the conclusiveness of the scores of the parameters is based on a combination of the assigned scores of the parameters corresponding to the first DNA sequencing technique and the first knowledge score, or on a combination of the assigned scores of the parameters corresponding to the second DNA sequencing technique and the second knowledge score.

19. The method of claim 17, wherein a combined score between a first threshold value and second threshold value is determined as inconclusive, a combined score below a first threshold value is determined as conclusively untrue, and a combined score above a second threshold value is determined as conclusively true.

20. The method of claim 11, wherein the third sequencing method includes didoxynucleotide chain termination (Sanger) sequencing.
